(19) United States
(12) Patent Application Publication
(10) Pub. No.: US 2006/0030781 A1
(43) Pub. Date: Feb. 9, 2006

(54) Emergency heart sensor patch

(76) Inventor: Adnan Shennib, Dublin, CA (US)

Correspondence Address: GLENN PATENT GROUP, 3475 EDISON WAY, SUITE L, MENLO PARK, CA, 94025, US

(21) Appl. No.: 10/913,166

(22) Filed: Aug. 5, 2004

(51) Int. Cl. A61B 5/0402

(52) U.S. Cl. 600/509

(57) ABSTRACT

The invention provides a disposable sensor patch for the non-invasive detection and indication of a heart condition during a medical emergency. The patch is placed on the chest area for sensing and analyzing a surface electrocardiogram (ECG). The heart condition is rapidly indicated via an indicator integrated in the patch. The disposable smart patch is inexpensive and simple to use by a layperson assisting or living with the person experiencing the medical crisis. The patch is activated automatically upon its removal from the package and placement on the chest. The detection and indication occurs rapidly and within 90 seconds of placement on the chest. In another embodiment of the invention, a vibration sensor element is incorporated for detecting cardiovascular vibrations and for ruling out pulseless electrical activity.

### CROSS REFERENCE TO RELATED APPLICATIONS

This application is related to co-pending patent application entitled Heart Disease Detection Patch, filed jointly with this application, which application is incorporated herein in its entirety by the reference herein.

### BACKGROUND OF THE INVENTION

1. Technical Field

The invention relates to non-invasive electrocardiogram (ECG) monitoring. More particularly, the invention relates to heart condition detection during an emergency.

2. Description of the Prior Art

Cardiovascular diseases contribute to about 2.4 million deaths annually in the United States alone. Estimates of the number of heart attacks range from 1.2 to 1.5 million with 700,000 new cases reported annually. About 50% of deaths occur within one hour of a heart attack and prior to reaching a hospital. Heart attack symptoms are varied and can be confused with other ailments. Signs of a heart attack include discomfort in the chest, the stomach, and the neck, shortness of breath, nausea, light-headedness, and breaking into cold sweat. Although heart attacks are serious and can lead to death, the symptoms are too often unrecognized, leading to an otherwise preventable death. Seeking prompt medical help, particularly during the first 30 minutes of a heart attack is considered critical for the outcome of a heart attack. This is particularly the case in dealing with blood clots formed during a myocardial infarction (MI), a major form of a heart attack For survivors, delay in recognition and treatment of a heart attack leads not only to damage to the heart tissue but also to other vital organs such as the brain. The consequence of delayed recognition and treatment also leads to higher cost of treatment and lower quality of life for the survivor.

Conversely, a false alarm due to symptoms similar but unrelated to the heart, leads many people to rush to emergency centers unnecessarily. This not only causes tremendous emotional and financial stress to the individual and the family, but also contributes to the stress on the health care system, which is already overwhelmed in many communities throughout the world. Therefore, improved and speedy diagnosis of a heart condition during a medical emergency not only saves lives but also reduces stress and cost to the health care system and society in general.

However, detection of heart conditions during an emergency remains problematic with conventional methods and instruments. Pulse detection, a basic indicator of heart activity, is often inadequate for assessing the possibility of a heart attack. The average person, e.g. spouse, family member, friend, or a bystander, assisting the affected person may not be skilled in the art of pulse palpation. The emergency situation can also make pulse detection more difficult due to the emotional stress or the environment of the occurrence. Even if a pulse is detected, assessment of the cardiac condition based on heart rate alone is inadequate in view of possible abnormalities.

The non-invasive sensing of surface potentials of cardiac electrical activity, i.e. the electrocardiogram (ECG), remains one of the most reliable and effective methods for proper diagnosis of cardiac function. Conventional ECG methods involve attaching electrodes to the body, mostly on or near the heart area on the chest, and connecting electrode wires (cables) to an electronic instrument having a monitor that displays the ECG waveform. Key parameters, such as heart rate, are normally displayed as well.

Heart abnormalities are generally visually observed by medical personnel skilled in ECG interpretation. ECG interpretation can also be automated by a microprocessor (processor) incorporated within the ECG instrument. The cost, bulk, and complexity of standard ECG instruments render its application impractical outside medical settings. Unfortunately, most emergencies occur outside medical settings, such as at home, businesses, restaurants, and vehicles.

Holter monitors are specialized instruments for long term ECG monitoring at home (for example see U.S. Pat. No. 6,456,872 to Faisandier). These instruments use five or more ECG electrodes attached to the chest at one end and to a portable device on the other end. The device is worn or strapped to the body for recording ECG signals in its memory. Holter monitors may also incorporate an alarm for warning the patient of an adverse cardiac event. After 24 or 48 hours of monitoring the Holter monitor is typically returned to the clinic, where the recorded ECG data are downloaded for viewing, record keeping and for further analysis if necessary. Trans-telephonic data transmission of ECG data is also widely employed for individuals who require longer term or daily monitoring of ECG. Holter monitors and other portable ECG instruments are also relatively expensive, cumbersome, and offered only to select patients as prescribed by a physician.

Cardiac event recorders are hand-held ECG devices with integrated electrodes for instant momentary self-application of the device on the chest whenever a cardiac event is suspected, i.e. heart palpitation, dizziness, chest pain. Conventional ECG event recorders are rugged reusable devices and record only a few minutes of ECG data.

There are also a variety of non-medical consumer-oriented heart and pulse monitors available for wellness and fitness applications. These are offered in the form of wristwatch, belt-worn, and pocket-worn devices. These devices may have built-in electrodes or may be supplied with cable-connected electrodes for sensing and computing ECG parameters, such instantaneous and average heart rate. Although considerably less expensive than Holter monitors and ECG event recorders, these monitors offer little diagnostic capability and, thus, are not suitable for medical applications.

There is a need for a simple, reliable and low cost device for heart condition detection and indication. However, prior art instruments and methods fall short in achieving the objectives of this invention stated below.

For example, U.S. patent application No. 2003/0069510 to Semler discloses a disposable vital signs monitor in the form of a patch having a “flexible, nominally flat planer form having integral gel electrodes, a sticky-back rear surface, an internal flex circuit capable of sensing, recording, and play out several minutes of the most recently acquired ECG waveform data and a front surface that includes an output port preferably having one or more snap connectors compatible with lead harness . . . . ” This playback and analysis is presumably performed in a medical setting under the supervision of skilled medical personnel. In another embodiment of Semler's invention, the monitor is remotely controlled by telemetry and is capable of delivering pacer or defibrillation pulses to the patient. Although inexpensive as a disposable ECG event recorder, it offers no integral analysis or indication of the heart condition. Therefore, Semler's invention has limited application, if any, for the assessment of a person's heart condition during an emergency.

U.S. patent application No. 2003/0083559 to Thompson discloses a peripheral monitor patch for attachment to a patient including high capacity memory for storage and later retrieval of the recorded ECG data. The patch comprises novel non-contact electrodes. The patch neither provides diagnostic capability, nor indication of heart condition.

U.S. Pat. No. 6,690,959 to Thompson discloses a smart patch with nano-spikes for improving the electrode-skin contact. The '959 invention does not provide built-in diagnostic and indicators to detect and indicate a heart condition, and thus is not suitable for dealing with medical emergencies.

Kagan et al in U.S. Pat. No. 5,443,072 disclose a disposable blood flow monitor which is adhered directly to the skin above the vessel to be monitored. Kagan's invention does not deal with analysis or indication of heart function, and thus is not suitable during a medical emergency.

Hagan et al in U.S. Pat. No. 6,572,636 discloses a pulse sensing patch with an indicator for displaying a visually recognizable pattern of detected pulses. As discussed above, pulse detection provides in adequate diagnosis during a medical emergency.

It would be advantageous to provide a disposable low cost non-invasive heart condition detector and indicator for use in a medical emergency.

It would be further advantageous to provide an automatic heart function test that is simple to self-administer or be administered by a layperson assisting a person experiencing a medical crisis.

It would be further advantageous to provide detection and indication of a heart condition rapidly, e.g. and within 90 seconds.

It would be further advantageous to provide interim heart monitoring means until the arrival or presence of medical personnel.

It would be further advantageous to provide an inexpensive miniature cardiac sensor for use in first aid kits.

It would be especially advantageous to provide rapid detection of a heart attack during a medical emergency.

### SUMMARY OF THE INVENTION

The invention provides a disposable sensor patch for the non-invasive detection and indication of a heart condition during a medical emergency. The patch is placed on the chest area for sensing and analyzing surface electrocardiogram (ECG). The smart patch automatically obtains and analyzes ECG waveform and searches for abnormalities, particularly those that are heart attack related. The heart condition is rapidly indicated via an indicator integrated in the patch. The smart emergency patch is designed for simple intuitive use by a layperson assisting or living with the person experiencing the medical crisis. The patch may also be self administered if the affected person is sufficiently cognizant. The patch is preferably activated automatically upon its removal from the package and placement on the chest. In one embodiment, the status of the heart is indicated via multiple LEDs. The detection and indication occurs rapidly, e.g. within 90 seconds of placement on the chest. This allows the affected person, or the layperson assisting, to make the necessary decisions, with regard to treatment, assistance, and the like, which are often critical for the outcome of the emergency event.

The smart cardiac patch is thin, flexible, and incorporates a battery, ECG amplifier, electrodes, indicator, and a processor for analyzing ECG the waveform and detecting and indicating the heart condition. The smart patch is a highly integrated electronic assembly, designed for mass production, and is thus inexpensive and suitable for disposable use. During a medical crisis, the integrated processor and associated software algorithm, automatically searches for a cardiac abnormality, such as arrhythmia, bradycardia, tachycardia, fibrillation, mycocardial infarction, ischemia, premature ventricular contractions (PVCs), premature atrial contractions (PACs), blocks or pulseless electrical activity (PEA). In another embodiment of the invention, a vibration sensor element is incorporated for detecting cardiovascular vibrations, such as heart sounds and blood flow. The vibration sensor is particularly useful in detecting PEA with symptoms of mechanical inactivity and sometimes ECG within normal range. The cardiac sensor patch may also be placed on the neck area for ease of access in an emergency situation and for proximity to a pulsating carotid artery.

The application of this disposable cardiac sensor patch is broad and ranges from home and office first aid kits, living establishments of high-rise individuals, such as nursing homes, sports and public arenas, to medical centers. The emergency heart sensor patch is particularly designed for short term or spot check applications during a medical emergency.

In addition to rapid detection and indication, additional heart monitoring is preferably provided for at least 15 minutes. This is to provide continuous monitoring until the arrival of medical personnel or until resolution of the medical crisis. In another embodiment, memory is offered for recording of ECG data, particularly of abnormal events. Stored ECG data are later retrieved in a clinical setup by an interrogation device. This feature is intended to provide a record of transient cardiac events, which often become illusive for medical personnel to subsequently detect and document.

### BRIEF DESCRIPTION OF THE DRAWINGS

FIG. 1 is a view of the emergency heart monitor patch placed on the chest of a person experiencing a medical emergency;

FIGS. 2(a-c) is a typical ECG sequence pattern during a myocardial infarction heart attack;

FIG. 3 is a top view of the emergency heart monitor patch showing four electrodes, flexible circuit, battery and other major components;

FIG. 4 is a cross section view of the emergency monitor patch of FIG. 3, showing the various layers with thickness exaggerated for clarity;

FIG. 5 shows a rectangular embodiment of the emergency cardiac monitor patch with four electrodes and vibration transducer for sensing cardiovascular vibrations;

FIG. 6 shows a two-electrode band-shaped embodiment with vibration sensor made of a piezoelectric film;

FIG. 7 shows the band patch embodiment of FIG. 6 placed on the chest for sensing surface ECG and heart vibrations;

FIG. 8 shows the embodiment of FIG. 6 placed on the neck for sensing surface ECG and pulse activities near the carotid artery; and

FIG. 9 shows optical transmission of ECG data, recorded by the emergency heart monitor patch, to an external device.

### DETAILED DESCRIPTION OF THE INVENTION

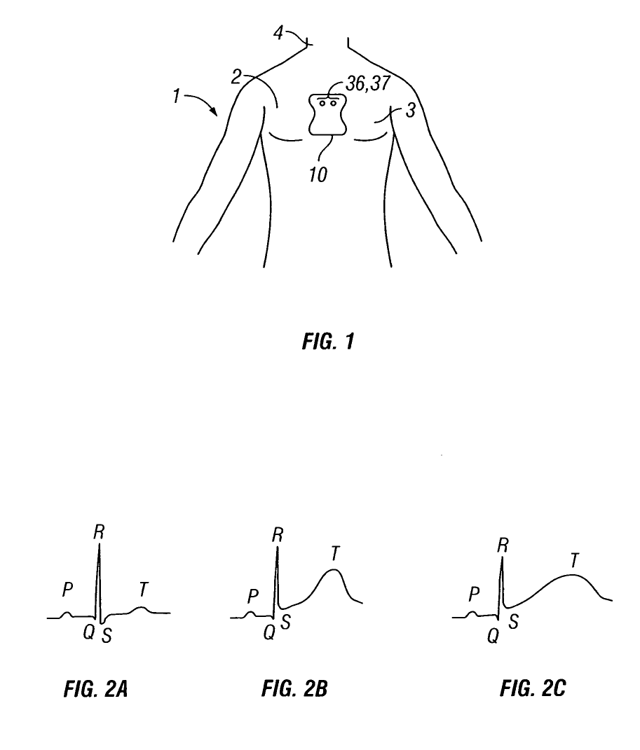

The invention, shown in various embodiments of FIGS. 1 and 3-9 is a non-invasive cardiac patch for detection and indication of a heart condition during a medical emergency. The patch 10 is thin and flexible for placement on the chest area 2 of a person 1 experiencing a medical emergency, particularly with symptoms of a heart attack The sensor patch relies on a surface electrocardiogram (ECG) for detecting and analyzing non-invasively the electrical activity of the heart and indicating the results through an indicator integrated into the patch. The smart patch is fully self-contained and self-powered.

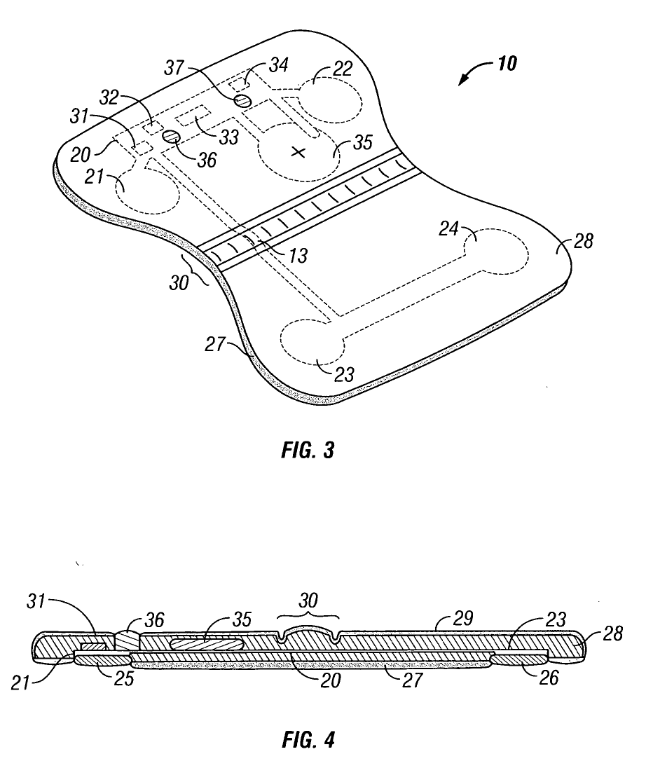

Referring to the embodiment of FIGS. 3 and 4, the emergency patch 10 comprises, for example, four ECG electrodes 21, 22, 23 and 24, an ECG amplifier 31, a processor 33, and a battery 35. The processor 33 is typically a digital signal processor for performing numerical computation from data obtained from an analog-to-digital converter 32. The sensor patch 10 also incorporates a memory 34, referring generally here to all types of electronic memory for storage of program data and acquired ECG data, if so desired.

The electronic assembly of the invented patch is formed of a flexible circuit substrate 20 with trace extensions to the electrodes 21, 22, 23, 24 and to the battery 35. A conductive gel 25, 26 (FIGS. 3 and 4) covers the electrodes 21, 22, respectively, as well as other electrodes not shown in the view of FIG. 3. The conductive gel 25 and 26 contacts the skin directly to conduct surface ECG potentials to the electrodes and the ECG amplifier 31. The electrodes may be pre-gelled as shown or alternately made for dry contact (not shown) with electrodes directly contacting the skin. A non-conductive pad 27 provides skin contact, preferably made of gel or an adhesive, i.e. Hydrogel, for adhesion to the skin. Alternatively, the padding may be made of a low-durometer rubber or elastomeric material. The patch 10 also comprises a thin substrate 28 for providing structural support. The substrate 28 is made of soft and flexible material, such as polyurethane or cloth. A pad 27 and a substrate 28 may be of the same material to reduce the cost of manufacture. The thickness of the patch device 10 (shown not to scale for clarity) is preferably in the range of 1.5 and 2.5 mm, but preferrably no more than 3.5 mm. A groove area 30 and a trace loop 13 provide additional flexibility for a folding area that is provided for use when storing the patch 10 while in its package.

In the preferred embodiment, the smart heart monitor patch 10 comprises two to four ECG electrodes, depending on ECG results desired. FIGS. 3-5 show a four-electrode embodiment for placement on the chest area. The electrodes are arranged to provide a modified three-lead configuration with the electrodes 21, 22, 23, 24 representing right arm (RA), left arm (LA), right leg (RL), and left leg (LL) leads. This configuration produces standard bipolar leads Lead-I, Lead-II, and Lead-III, as well as augmented leads aVR, aVL and aVF. Multi-lead configurations provide improved accuracy in detecting cardiac abnormalities.

Cardiac abnormalities range from simple heart rate arrhythmias to complex waveform patterns requiring detailed analysis and pattern recognition. For example, a heart rate exceeding 160 beats per minute (BPM) at rest indicates a tachycardia condition which may soon evolve to fibrillation and death within minutes if not treated promptly. On the other hand, a heart rate of 45 BPM or below indicates a low rate or bradycardia, a serious but not necessarily a fatal condition. A cardiac arrest condition requires immediate application of cardiopulmonary resuscitation (CPR) therapy. A cardiac arrest is evident by absence of ECG or key features of an ECG.

The invention provides waveform analysis which is critical in determining abnormalities that are not detectable by standard pulse detection (heart rate) methods. For example, a person experiencing a heart attack may exhibit a pulse rate well within the normal range. However, analysis of the ECG waveform may reveal a serious heart condition. The smart patch of the invention automatically provides real-time analysis of the ECG waveform and rapidly indicates an abnormality, particularly related to a heart attack The software algorithm executed by processor 33 searches for cardiac abnormalities, including arrhythmia, bradycardia, tachycardia, fibrillation, mycocardial infarction, ischemia, premature contractions, blocks, or pulseless electrical activity (PEA).

FIG. 2(a-c) shows a typical ECG pattern for a person experiencing a heart attack caused by a myocardial infarction (Ml). Briefly described here, when the blood supply is abruptly reduced or cut off to a region of the heart, a sequence of injuries may occur beginning with transmural ischemia, followed by necrosis and eventually fibrosis (scarring) if the blood supply to the affected area is not restored in a timely manner. FIG. 2a shows, for reference purposes, normal ECG consisting of a P wave, QRS complex, and T-wave. FIG. 2b. shows an early sign of Ml indicated by a sharp increase in the amplitude and width of the T-wave. As Ml progresses, the T-wave generally broadens further with elevation of the ST-segment as shown in FIG. 2c, indicating the likely occurrence of transmural injury. This and other abnormalities, particularly in the acute stage, exhibit patterns that are well recognized in the field of cardiovascular disease and electrophysiology.

Heart condition indication is provided by the on-board indicator 36. In the embodiment shown in FIGS. 1 and 3, two light emitting diode (LED) indicators 36 and 37 are provided in two different colors. For example, a green LED light indicates a safe heart condition while a red LED light indicates a risk condition. The LEDs can also be used to indicate general heart activity during collection of data and prior to indicating a heart condition. For example, one or two of the LEDs can be flashing in synchrony with QRS pulses immediately upon placement of the smart patch on the body and upon the detection of an ECG waveform. Later on, upon completion of the analysis, e.g. within 90 seconds, or sooner if properly detected, either the green or red LED is activated depending on the results of the analysis of the ECG waveform. A serious cardiac condition may be determined well before 90 seconds, and thus is indicated promptly upon collecting sufficient data for determining the adverse condition. For example ten to fifteen seconds are sufficient to properly detect cases of fibrillation, acute myocardial infarction, or cardiac arrest.

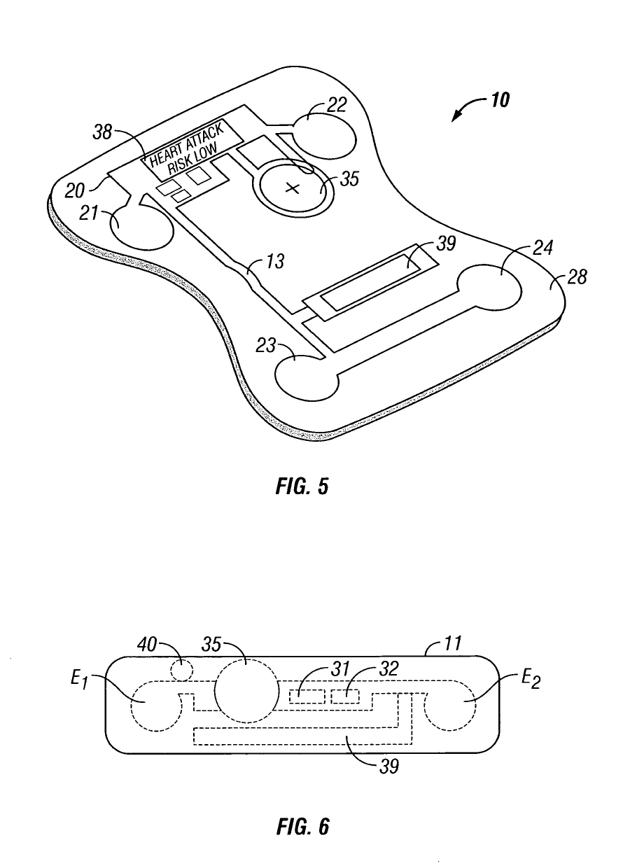

Other possible indicators include audible transducers, such as a buzzer (not shown) or a speaker (not shown), and other visual indicator types, such as a liquid crystal display (LCD) 38 as shown in FIG. 5. Electrochemical indicators (color strips) are also envisioned. The advantage of an LCD or multi-color indicator is the ability to indicate different levels of risks, such as providing the words “normal” or “risk.” An LCD can actually spell out the condition and the course of action desired for example by displaying statements such as, “heart attack,” “see doctor,” CPR required,” or “dial 911.” An LCD indicator can actually spell out the condition to communicate accurately to remote medical staff if necessary. For example, the caller can specifically communicate a fibrillation condition to a 911 operator, thus focusing the efforts on seeking defibrillation means. Presently, LCD technology is sufficiently thin and miniature to be easily integrated in the invention.

One key feature of the invention is integrating in a single low cost disposable patch the combination of heart condition detection and indication. This is enabled by the application of highly integrated battery operated electronic assembly with built-in algorithms for ECG waveform detection, analysis, and indication. The need for expensive ECG monitors and skills for analysis and interpretation are therefore substantially mitigated with the invention during an emergency. The level of cost reduction, ease-of-use, and integration provided by the invention allow for availability and accessibility not possible with prior art systems.

Although most adverse cardiac conditions are detected solely from ECG signal, detection of certain adverse conditions can be enhanced by the application of a secondary sensor. FIG. 5 shows a four-electrode embodiment with a vibration sensor 39 for sensing cardiovascular vibrations, including heart sounds, on the surface of the body. This is particularly useful in establishing or ruling out pulseless electrical activity (PEA) condition, formerly know as electromechanical dissociation (EMD). PEA is a condition whereby the heart is mechanically stopped while exhibiting electrical activity. PEA may occur in individuals experiencing hypothermia or late stage chronic heart disease. In the embodiment of FIG. 5, cardiovascular vibrations including blood flow sounds can be detected by a vibration sensor 39. An amplifier (not shown) is necessary to amplify the electrical output of the vibration sensor 39. In the preferred embodiment, the vibration sensor 39 is a piezoelectric film. Other possible vibration transducers include an electret element, a silicone micro-electro-mechanical (MEM) element, an electromagnetic coil and other elements used in microphonic systems.

FIG. 6 shows a band-shaped patch 11 with a two-electrode embodiment, E₁ and E₂, for sensing the surface ECG. A vibration sensor 39 may also be incorporated as shown for sensing cardiovascular vibrations. A multi-color LED 40 is used to indicate heart activity and condition.

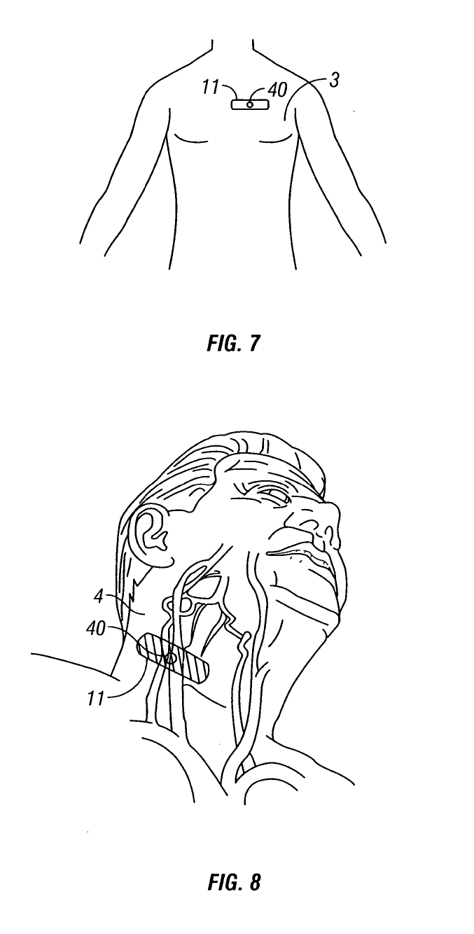

The elongated band embodiment of FIG. 6 is compact and suitable for placement on the heart area 3 as shown in FIG. 7. Alternatively, the band patch 11 may be positioned on the neck area 4 for sensing pulses and blood flow vibrations of either the left or right carotid artery on the neck (right side shown in FIG. 8).

Various filtering methods are known in the field of signal processing and particularly pertaining to ECG signals. Signal averaging of multiple ECG periods may be used to enhance the details of ECG features. Signal processing is also used for filtering and minimizing noise present in the ECG. For example, notch filters are effective in removing 60-Hz noise present in the environment. To minimize electromagnetic interference, a metal foil 29 (FIG. 4) is provided over the patch, entirely, or selectively over components sensitive to the interference.

In addition to rapid detection and indication, additional continuous heart monitoring is preferably provided for at least fifteen minutes. This is to provide continuous monitoring until the arrival of medical personnel, or until resolution of the medical crisis. In yet another embodiment enabled by the invention, the smart patch is used for detecting an adverse heart condition during stress which may be physical or psychological. For example, individuals with known heart conditions may temporarily wear the patch to detect and alert to an adverse heart condition prior to developing into a serious heart condition, for example, while performing a physical activity. In a similar related application, the person may wear the patch during a psychologically stressful condition that may adversely affect the individual's heart condition.

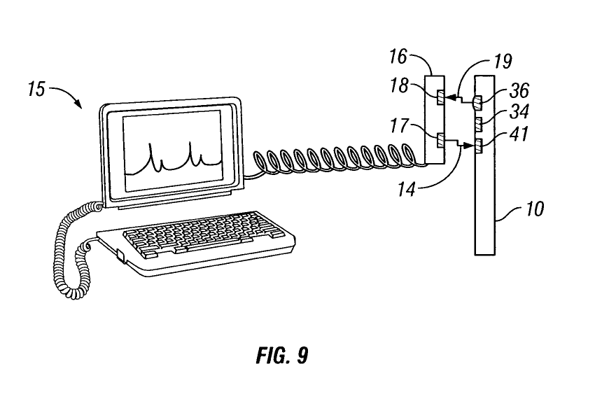

In another embodiment, a memory 34 is provided for automatic recording of abnormal ECG events. This feature provides a record of transient cardiac events which often become illusive for medical personnel to detect and document subsequently. The recorded ECG data is later retrieved by an interrogation device 15 (FIG. 9) in the clinic. The transmission of data preferably uses existing components to reduce cost and complexity of the disposable patch. For example, FIG. 9 (shown not to scale) shows optical transmission 19 of ECG data using the LED indicator 36 incorporated within the emergency cardiac patch 10. In this embodiment, ECG data are transmitted from the LED indicator 36 to an optical receiver 18 incorporated in the interrogation interface 16 of the external device 15. The activation of the data transmission is preferably automatic. For example, a magnetic field 14 from a magnet 17 within the interface 16 triggers an activation sensor 41, i.e. a reed-switch, within the patch to initiates data transmission. Activation can also be by manual means, such as by pressing an electromechanical switch incorporated onto the flexible substrate 20.

The wireless transmission of biologic data including detection results, ECG, and cardiovascular sounds may be accomplished in numerous ways and methods known in the field of medical devices and wireless data transmission. This includes optical means as shown above, or radio frequency (RF), magnetic, ultrasonic, and acoustic transmission. Inductive coupling through a coil (not shown) can also be used to transmit biologic data, as well as for powering the patch remotely during data transmission.

Proper adhesion to the skin is important for securing the patch to the person during the emergency event. Because the smart patch is intended only for temporary short-term periods, weak adhesion is sufficient. Furthermore, proper electrode-skin contact is desirable for obtaining adequate ECG signal-to-noise-ratio. Proper electrode-skin contact can be determined automatically and indirectly by measuring the impedance between adjacent electrodes. Normal electrode-electrode impedance for closely positioned electrodes is generally well under 10 LC-ohms depending on the condition of the skin and the distance between the electrodes. Measurement and detection of electrode-electrode impedance can also be used to activate the patch device 10 automatically upon its placement on the skin. Automatic activation can also b e accomplished during the removal of the patch device 10 from its package, i.e. a pouch, for example by incorporating open-circuit and/or short-circuit conditions between the electrodes within the package. These circuit conditions are altered during the removal of the patch device 10 from the package triggering the activation of the device. These and other automatic activation means and methods will be readily recognized by those skilled in the art of electronics and medical device packaging.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A disposable cardiac patch for non-invasive cardiac sensing of a person experiencing a medical emergency, said disposable patch comprising:
at least two electrodes for contacting a person's skin surface, said electrodes receiving a surface ECG signal;
an amplifier for amplifying said ECG signals from said electrodes;
a processor for performing real-time signal processing and analysis of said amplified ECG signals; and
means for detecting and indicating a heart condition of said person within a predetermined interval after applying said patch on the skin of said person.

2. The disposable patch o f claim 1, further comprises:
a power source for powering said disposable patch;
an indicator; and
a flexible substrate for incorporating said amplifier, said processor, said electrodes.

3. The disposable patch of claim 1, where said predetermined interval is 90 seconds.

4. The disposable patch of claim 1, wherein said patch has a thickness of less than 3.5 mm.

5. The disposable patch of claim 1, further comprising:
a flexible electronic circuit for interconnecting electronic components within said patch to said electrodes.

6. The disposable patch of claim 1, wherein said electrodes are configured to obtain any of Lead-I, Lead-II, and Lead-III ECG signal.

7. The disposable patch of claim 1, wherein said patch is placed on said person's chest area.

8. The disposable patch of claim 1, wherein said patch is placed in proximity to said person's heart.

9. The disposable patch of claim 1, wherein said patch is placed on said person's neck area.

10. The disposable patch of claim 1, further comprising
a memory for storing data comprising any of ECG signal and cardiovascular vibrations.

11. The disposable patch of claim 10, further comprising:
means for transmitting said data stored in said memory to an external device.

12. The disposable patch of device of claim 11, said means for transmitting data further comprising:
a transmitter associated with said patch, said transmitter comprising any of an optical element, an RF element, an inductive element, and an electromagnetic element.

13. The disposable patch of claim 11, further comprising:
means for activating said data transmission.

14. The disposable patch of claim 2, said indicator comprising an audio transducer.

15. The disposable patch of claim 2, said indicator comprising at least one visual indicator, comprising any of, a light emitting diode (LED), a color strip element, and a liquid crystal display (LCD).

16. The disposable patch of claim 15, wherein said visual indicator comprising a multicolored LED.

17. The disposable patch of claim 2, said substrate comprising:
a metal foil.

18. The disposable patch of claim 1, further comprising:
means for automatic powering and activation of said patch upon either of opening of a package containing said patch and placement of said patch on the skin of person.

19. The disposable patch of claim 1, further comprising an adhesive for adhering said patch to said person's skin.

20. The disposable patch of claim 19, said adhesive comprising a gel.

21. The disposable patch of claim 1, wherein said heart condition comprises any of bradycardia, tachycardia, fibrillation, arrhythmia, cardiac arrest, normal heart function, pulseless electrical activity, premature contraction, block and myocardial infarction.

22. The disposable patch of claim 1, said means for detecting a heart condition comprising a signal averaging means.

23. The disposable patch of claim 1, further comprising:
a vibration transducer for sensing cardiovascular vibrations and for producing a signal representative of said cardiovascular vibrations.

24. The disposable patch of claim 23, said vibration transducer comprising any of a piezoelectric film and an electret film.

25. The disposable patch of claim 23, wherein absence of said cardiovascular vibrations indicates a pulseless electrical activity (PEA).

26. The disposable patch of claim 23, wherein a heart condition is determined based on the detection and analysis of the combination of cardiovascular vibrations and, an ECG signal.

27. The disposable patch of claim 1, further comprising:
means for continuous heart monitoring and indication for at least 15 minutes.

28. A disposable patch for non-invasive monitoring of vital signs of a person experiencing a medical emergency, said patch comprising:
at least two electrodes for contacting said person's skin, said electrodes receiving a surface ECG signal;
an ECG amplifier for amplifying said ECG signal;
an indicator;
a flexible substrate for incorporating said amplifier, said processor, said electrodes, and said indicator into said patch;
a vibration transducer for sensing cardiovascular vibrations; and
means for rapidly detecting and indicating via said indicator a heart condition of said person.

29. The disposable patch of claim 28, further comprising:
a power source for powering said disposable patch.

30. The disposable patch of claim 28, wherein said heart condition is determined and indicated within 90 second of placing said patch on said person's skin.

31. The disposable patch of claim 28, wherein said heart condition comprises pulseless electrical activity (PEA).

32. The disposable patch of claim 28, wherein said patch is placed on said person's chest.

33. The disposable patch of claim 28, wherein said patch is placed on said person's neck.

34. A method for non-invasive cardiac sensing during a medical emergency, comprising the steps of:
placing a disposable patch on a persons skin, said person experiencing a medical emergency, said patch incorporating within an ECG amplifier, a processor, at least two electrodes for contacting said person's skin, and an indicator;
amplifying said ECG signal from said electrodes;
processing and analyzing ECG signal with said processor;
detecting a heart condition by said processor; and
indicating said heart condition by said indicator.

35. The method of claim 34, wherein said heart condition is indicated within 90 seconds of placing said patch on said person's skin.

36. The method of claim 34, wherein said heart condition comprises any of arrhythmia, bradycardia, tachycardia, fibrillation, cardiac arrest, myocardial infarction, PEA, normal heart function, premature contraction, block and heart failure.

37. The method of claim 34, wherein said for heart condition is indicated by visual display means comprising any of an LED and an LCD.

38. The method of claim 34, wherein said heart condition is indicated by an audible means.

39. The method of claim 34, further comprising the steps of:
storing data obtained by said disposable patch in a memory associated with said patch; and
transmitting said data to an external device.

40. The method of claim 34, wherein said disposable patch is placed on said person's chest area.

41. The method of claim 34, wherein said disposable patch is placed on said person's neck area.

42. A method for non-invasive vital sign detection and indication during a medical emergency, comprising the steps of:
placing a disposable patch on a person's skin, said person experiencing a medical emergency, said patch incorporating within a thin flexible substrate, an ECG amplifier, a processor, at least two electrodes for contacting said person's skin, a vibration transducer, and an indicator;
amplifying an ECG signal from said electrodes with said ECG amplifier and producing an amplified ECG signal;
detecting cardiovascular vibrations with said vibration transducer and producing an amplified vibration signal;
processing and analyzing amplified ECG signal and said amplified vibration signal with said processor; and
detecting a heart condition by said processor.

43. The method of claim 42, further comprising the step of:
indicating within 90 seconds of placing said monitor patch on the skin of the person the heart condition via said indicator.

44. The method of claim 42, wherein said heart condition comprises any of arrhythmia, bradycardia, tachycardia, fibrillation, cardiac arrest, PEA, myocardial infarction, normal heart function, premature contraction, block and heart failure.

45. The method of claim 42, wherein said disposable patch is placed on said person's chest area.

46. The method of claim 42, wherein said disposable patch is placed on said person's neck area.

47. A disposable cardiac patch for heart attack detection and indication comprising:
at least two electrodes for contacting a person's skin surface, said electrodes receiving a surface ECG signal;
an amplifier for amplifying said ECG signal from said electrodes;
a processor for performing real-time signal processing and analysis of said amplified ECG signal;
an indicator;
a flexible substrate for incorporating said amplifier, said processor, said electrodes, and said indicator into said patch; and
means for detecting a heart attack condition and activation of said indicator within 90 seconds of applying said patch on the skin of said person.

48. A disposable cardiac patch for detection of an adverse heart condition during a stress condition, said patch comprising:
at least two electrodes for contacting a person's skin surface, said electrodes receiving a surface ECG signal;
an amplifier for amplifying said ECG signals from said electrodes;
a processor for performing real-time signal processing and analysis of said amplified ECG signal;
an indicator;
a flexible substrate for incorporating said amplifier, said processor, said electrodes, and said indicator into said patch; and
means for detecting an adverse heart condition and activation of said indicator within 90 seconds of detecting said adverse heart condition.
